(19) United States
(12) Patent Application Publication
(10) Pub. No.: US 2026/0199850 A1
(43) Pub. Date: Jul. 16, 2026

(54) STIRRING APPARATUS

(71) Applicant: SHIMADZU CORPORATION, Kyoto (JP)

(72) Inventors: Motomu HINOOKA, Kyoto-shi (JP); Masaki KANEDA, Kyoto-shi (JP)

(73) Assignee: SHIMADZU CORPORATION, Kyoto (JP)

(21) Appl. No.: 19/379,705

(22) Filed: Nov. 4, 2025

(30) Foreign Application Priority Data
Jan. 15, 2025 (JP) 2025-005572

(51) Int. Cl. B01F 31/23; B01F 31/00; B01F 31/20; B01F 101/23; B01F 101/44; C12M 1/22; C12M 3/06

(52) U.S. Cl. CPC B01F 31/23; B01F 31/201; B01F 31/70; C12M 23/10; C12M 27/16; B01F 2101/23; B01F 2101/44

(57) ABSTRACT

A stirring apparatus comprises a mounting table 120 provided with a mounting surface 122 on which a container 110 housing an object to be stirred 111 is mounted; and a mounting table drive part 130, 140, 150, 160 that supports the mounting table 120 in a state where the mounting surface 122 is inclined with respect to a horizontal direction, and that continuously changes a direction of inclination of the mounting surface 122.

### TECHNICAL FIELD

The present invention relates to a stirring apparatus.

### BACKGROUND ART

In microbiological testing of food products and the like, the pour plate culture method is mainly applied as a culture method for examining the number of bacteria in a liquid sample. In the pour plate culture method, first, a predetermined amount of a sample is placed in a Petri dish, a liquid culture medium is then added thereto, and thereafter, the sample and the culture medium are mixed by shaking the Petri dish. Then, the Petri dish is allowed to stand still to solidify the culture medium, and the microorganisms in the sample are cultured by incubating at a predetermined temperature for a predetermined time.

Conventionally, in such a pour plate culture method, an operator has mixed (poured and mixed) the sample and the culture medium by moving the Petri dish, to which the sample and the liquid culture medium have been added, on a workbench so as to draw a circular trajectory or a figure-eight trajectory. In recent years, an automatic pour-mixing apparatus that automatically performs such a pour-mixing operation has also been developed (see, for example, Non-Patent Literature 1).

### PRIOR ART DOCUMENTS

### Non-Patent Literature

[Non-Patent Literature 1] “Fully Automatic Medium Dispensing and Pour-Mixing Apparatus MEDIA JET 360”, [online], Central Scientific Commerce, Inc., [searched on Jan. 15, 2025], Internet <URL: https://cscjp.co.jp/creos/mediajet360.html>

### SUMMARY OF THE INVENTION

### Problem to be Solved by the Invention

In the automatic pour-mixing apparatus described in Non-Patent Literature 1, the sample and the culture medium in the Petri dish are stirred and mixed by moving the Petri dish in a horizontal plane so as to draw a figure-eight trajectory. However, such an automatic mixing apparatus requires a relatively large space to move the Petri dish, which has resulted in a problem of an increase in the size of the apparatus.

The problem to be solved by the present invention has been made in view of the above points, and an object thereof is to downsize an apparatus that stirs an object to be stirred, which is housed in a container, by moving the container.

### Means for Solving the Problem

A stirring apparatus according to a first aspect of the present invention made to solve the above problem comprises:
a mounting table provided with a mounting surface on which a container housing an object to be stirred is mounted; and
a mounting table drive part that supports the mounting table in a state where the mounting surface is inclined with respect to a horizontal direction, and that continuously changes a direction of inclination of the mounting surface.

Furthermore, a stirring apparatus according to a second aspect of the present invention made to solve the above problem comprises:
a mounting table provided with a mounting surface on which a container housing an object to be stirred is mounted; and
a mounting table rotating part that autorotates the mounting table about an axis orthogonal to the mounting surface in a state where the mounting surface is inclined with respect to a horizontal direction.

### Advantageous Effects of the Invention

In the stirring apparatus according to the first aspect of the present invention having the above configuration, by inclining the container, the object to be stirred in the container is pressed against an inner peripheral surface of the container by gravity. Then, by changing the direction of inclination of the container in that state, a shear force is generated between a portion of the object to be stirred that is in contact with the inner peripheral surface of the container and the other portion, thereby stirring the object to be stirred. Furthermore, in the stirring apparatus according to the second aspect of the present invention having the above configuration, by inclining the container, the object to be stirred in the container is pressed against the inner peripheral surface of the container by gravity, and by autorotating the container in that state, a shear force is generated between the portion of the object to be stirred that is in contact with the inner peripheral surface of the container and the other portion, thereby stirring the object to be stirred. According to such a configuration, compared to a case where the container is moved so as to draw a circular trajectory (that is, a case of revolving the container) and a case where the container is moved so as to draw a figure-eight trajectory as in a conventional automatic pour-mixing apparatus, a space required for stirring can be reduced, and the apparatus can be downsized.

### BRIEF DESCRIPTION OF THE DRAWINGS

FIG. 1 is a schematic diagram showing a schematic configuration of a stirring apparatus according to a first embodiment of the present invention.

FIG. 2 is a schematic diagram showing a configuration of a rotation restricting part in the stirring apparatus.

FIG. 3 is a schematic diagram for explaining the movement of the stirring apparatus.

FIG. 4 is a schematic diagram showing another configuration example of the stirring apparatus according to the first embodiment.

FIG. 5 is a schematic diagram showing still another configuration example of the stirring apparatus according to the first embodiment.

FIG. 6 is a schematic configuration diagram of a stirring apparatus according to a second embodiment of the present invention.

### DESCRIPTION OF EMBODIMENTS

### Embodiment 1

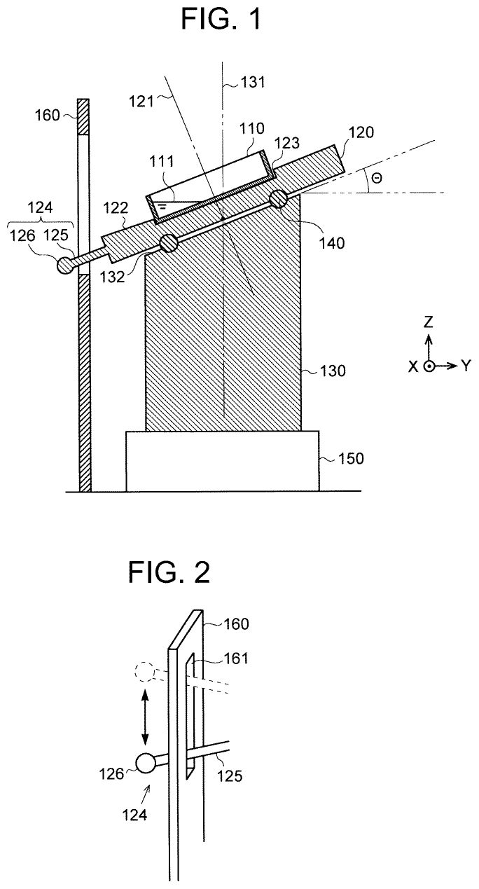

A first embodiment of the present invention will be described with reference to the drawings. FIG. 1 is a diagram showing a schematic configuration of a stirring apparatus according to the present embodiment. In the figure, some components are shown in cross-section for convenience. The stirring apparatus according to the present embodiment stirs a liquid 111 housed in a Petri dish 110 (corresponding to a container in the present invention), and comprises a mounting table 120 on which the Petri dish 110 is mounted, a mounting table support part 130 that supports the mounting table 120 from below, a connecting part 140 that connects the mounting table support part 130 and the mounting table 120, a rotational drive part 150 that rotates the mounting table support part 130 about a central axis 131 extending in a vertical direction, and a rotation restricting part 160 that restricts the rotation of the mounting table 120. Here, the mounting table support part 130, the connecting part 140, the rotational drive part 150, and the rotation restricting part 160 cooperate to function as a mounting table drive part in the present invention. Hereinafter, for convenience of explanation, the arrow direction of the Y-axis in FIG. 1 is defined as the rightward direction, the arrow direction of the X-axis as the forward direction, and the arrow direction of the Z-axis as the upward direction, and front-rear, up-down, and left-right are defined.

The mounting table support part 130 is a columnar member extending in the up-down direction, and an upper end surface thereof is an inclined surface that is inclined by a predetermined inclination angle Θ with respect to a horizontal plane. This inclined surface is hereinafter referred to as a support surface 132 (corresponding to a mounting table support surface in the present invention). A lower end of the mounting table support part 130 is connected to the rotational drive part 150.

The rotational drive part 150 includes a drive mechanism such as a motor (not shown), and autorotates the mounting table support part 130 about its central axis 131 (corresponding to “a vertical axis passing through the mounting table support surface” in the present invention).

The mounting table 120 is a plate-shaped member, and a circular recessed part 123 for fitting the Petri dish 110 is provided in the center of an upper surface thereof (hereinafter, referred to as a mounting surface 122). An engaging part 124 that engages with the rotation restricting part 160 protrudes from a peripheral surface of the mounting table 120 (details will be described later). As shown in FIG. 1, the mounting table 120 is disposed on the support surface 132, which is an inclined surface. Therefore, the mounting surface 122 is in a state of being inclined with respect to the horizontal plane at the same inclination angle Θ as the support surface 132. Here, it is desirable that the inclination angle Θ be, for example, 0° to 30°. As will be described later, the direction of inclination (downward direction) of the support surface 132 changes periodically with the rotation of the mounting table support part 130, and accordingly, the direction of inclination of the mounting surface 122 also changes periodically.

A connecting part 140 is provided between the support surface 132 of the mounting table support part 130 and a lower surface of the mounting table 120. The connecting part 140 is composed of, for example, a bearing such as a ball bearing, and by this connecting part 140, the mounting table 120 and the mounting table support part 130 are connected so as to be rotatable relative to each other about a central axis 121 of the mounting table 120 (that is, an axis perpendicular to the support surface 132).

The rotation restricting part 160 is a plate-shaped member provided with a slit 161 extending vertically as shown in FIG. 2, and is installed in an upright posture in the vicinity of the mounting table support part 130. The engaging part 124 provided on the mounting table 120 is engaged with the rotation restricting part 160. The engaging part 124 comprises a rod-shaped part 125, which is a round bar protruding perpendicularly from the peripheral surface of the mounting table 120, and a spherical retaining part 126 provided at a tip of the rod-shaped part 125. The rod-shaped part 125 has a diameter smaller than the width of the slit 161 of the rotation restricting part 160, and is inserted into the slit 161. The retaining part 126 has a diameter larger than the width of the slit 161, and is disposed on the opposite side of the mounting table 120 across the rotation restricting part 160. As a result, the engaging part 124 is movable in the longitudinal direction of the slit 161 and immovable in the width direction of the slit 161.

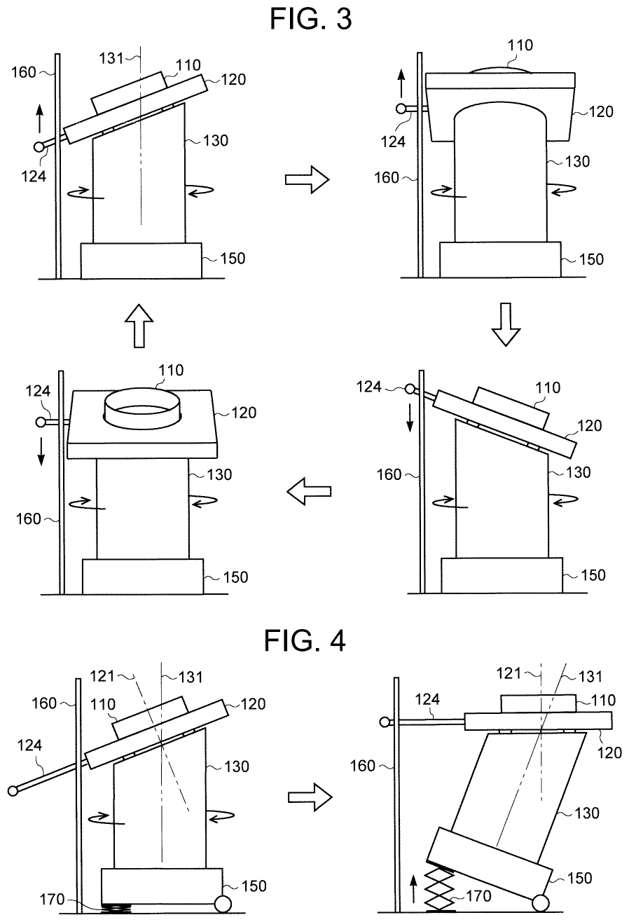

The operation of the stirring apparatus according to the present embodiment will be described with reference to FIG. 3. First, a user mounts a Petri dish 110 housing a liquid 111 to be stirred (for example, a liquid sample containing microorganisms and a liquid culture medium) on the mounting table 120. At this time, the Petri dish 110 is fitted into the recessed part 123 provided in the mounting table 120. Subsequently, the user operates a switch or the like (not shown) to actuate the rotational drive part 150. As a result, the mounting table support part 130 starts to rotate about its central axis 131. At this time, the mounting table support part 130 rotates, for example, clockwise as shown in FIG. 3, and accordingly, the state of the stirring apparatus changes as indicated by the white outlined arrows in the figure.

That is, assuming that the state in the upper left of FIG. 3 is an initial state, as the mounting table support part 130 rotates clockwise from the initial state, the support surface 132 and the mounting table 120 thereon change from a state of being inclined so as to become lower toward the left, as shown in the upper left of FIG. 3, to a state of being inclined so as to become lower toward the rear, as in the upper right of the figure. Subsequently, the support surface 132 and the mounting table 120 thereon pass through a state of being inclined so as to become lower toward the right, as in the lower right of the figure, and change to a state of being inclined so as to become lower toward the front, as in the lower left of the figure, and thereafter, return to the initial state again (note that the above-mentioned inclination angle Θ is kept constant in any state). That is, with the rotation of the mounting table support part 130, the direction of inclination of the support surface 132 and the mounting table 120 changes continuously and periodically in a clockwise direction, and accordingly, the direction of inclination of the Petri dish 110 mounted on the mounting table 120 also changes continuously and periodically in a clockwise direction. Here, it is desirable that a period of rotation of the mounting table support part 130 (that is, a period of change in the direction of inclination of the support surface 132, the mounting table 120, and the Petri dish 110) be, for example, 0.5 seconds to 3 seconds. As described above, since the engaging part 124 is immovable in the width direction of the slit 161 due to the engagement with the rotation restricting part 160, the mounting table 120 does not rotate even when the support surface 132 rotates. In this way, in the stirring apparatus according to the present embodiment, by inclining the Petri dish 110, the liquid 111 in the Petri dish 110 is pressed against an inner peripheral surface of the Petri dish 110 by gravity. Then, by changing the direction of inclination of the Petri dish 110 in that state, a shear force is generated between a portion of the liquid 111 in the Petri dish 110 that is in contact with an inner wall surface of the Petri dish 110 and other portions, whereby the liquid 111 is stirred.

Although the mounting table support part 130 is rotated clockwise in the above description, it may, of course, be rotated counterclockwise. Furthermore, clockwise rotation (forward rotation) and counterclockwise rotation (reverse rotation) may be alternately repeated at predetermined time intervals. As a result, the forwardly rotated liquid 111 and the reversely rotated liquid 111 can be made to collide, and the liquid 111 in the Petri dish 110 can be mixed more effectively.

As described above, in the stirring apparatus according to the present embodiment, since the liquid 111 in the Petri dish 110 is stirred by changing the direction of inclination of the Petri dish 110, a space required for stirring can be reduced compared to a conventional case where a container is moved so as to draw a circular or figure-eight trajectory, and the apparatus can be downsized. Furthermore, in the conventional apparatus, it is necessary to use a two-axis orthogonal stage to move the Petri dish 110 so as to draw a circular or figure-eight trajectory, and it is necessary to synchronize the two axes, which has resulted in a problem of complicated control. Furthermore, the adjustment of control parameters becomes complicated, and if the parameters are not appropriate, there has been a risk that the liquid may spill out of the Petri dish. In contrast, the stirring apparatus according to the present embodiment can be realized by a simple mechanism of a single-axis drive system, and the adjustment of parameters is also simple.

The stirring apparatus according to the present embodiment may be configured such that the mounting table 120 temporarily assumes a horizontal posture in the stirring process. A configuration example in this case is shown in FIG. 4. In this configuration example, a jack-shaped vertical drive mechanism 170 for vertically moving a left end of the rotational drive part 150 is provided at a lower part of the rotational drive part 150. Then, during stirring by the stirring apparatus, as shown on the left side of FIG. 4, when the mounting table 120 is in a state of being inclined so as to become lower toward the left (that is, the above-mentioned initial state), the rotation of the mounting table support part 130 is temporarily stopped. Thereafter, as shown on the right side of FIG. 4, the left end of the rotational drive part 150 is raised by the vertical drive mechanism 170 until the central axis 121 of the mounting table 120 becomes parallel to a vertical axis, thereby bringing the mounting table 120 into a horizontal posture. Thereafter, the left end of the rotational drive part 150 is lowered by the vertical drive mechanism 170 to bring the mounting table 120 into an inclined posture, and the rotation of the mounting table support part 130 by the rotational drive part 150 is restarted. At this time, the direction of rotation of the mounting table support part 130 is made to be opposite between before the mounting table 120 is made horizontal and after it is made horizontal. As a result, the liquid 111 in the Petri dish 110 can be stirred more effectively, and by temporarily bringing the mounting table 120 into a horizontal posture as described above, it is possible to prevent the liquid 111 in the Petri dish 110 from spilling due to the collision between the forwardly rotated liquid 111 and the reversely rotated liquid 111.

Although the mounting table 120 is described as being rectangular and plate-shaped in FIG. 3, the shape of the mounting table 120 is not limited to this, and may be, for example, disk-shaped or the like.

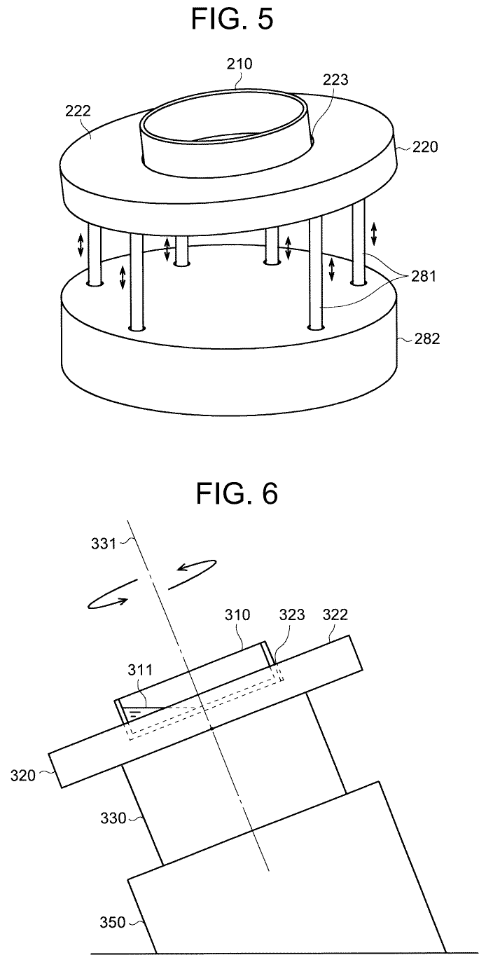

Furthermore, the stirring apparatus according to the present embodiment may be anything that changes the direction of inclination of the mounting table 120, and may have, for example, a configuration as shown in FIG. 5. In the figure, components that are the same as or correspond to those shown in FIG. 1 are denoted by reference numerals whose last two digits are common, and a description thereof will be omitted as appropriate. The stirring apparatus shown in the figure comprises a mounting table 220 provided with a recessed part 223 similar to the above, and a plurality of elevating/lowering parts 281 and an elevating/lowering part drive part 282 for changing the direction of inclination of the mounting table 220. Each elevating/lowering part 281 is a rod-shaped member extending vertically, and an upper end of each elevating/lowering part 281 is connected to positions on a lower surface of the mounting table 220 that are spaced apart from each other in a horizontal direction. A lower end of each elevating/lowering part 281 is connected to the elevating/lowering part drive part 282. The elevating/lowering part drive part 282 comprises a motor and a conversion mechanism (neither is shown) that converts the rotation of the motor into vertical movement of each elevating/lowering part 281, and is configured to sequentially move the plurality of elevating/lowering parts 281 up and down in a clockwise or counterclockwise direction. Even in such a configuration, the direction of inclination of the mounting table 220 can be changed continuously and periodically. As a result, the liquid in the Petri dish 210 can be rotated within the Petri dish 210 while being pressed against an inner peripheral surface of the Petri dish 210 by gravity, and the mixing of the liquid by the above-mentioned shear force can be realized. Even in such a configuration, it is desirable to control each elevating/lowering part 281 so that the inclination angle of the mounting table 200 with respect to the horizontal plane is kept constant. As a result, the liquid in the Petri dish 210 can be effectively prevented from spilling. Here, the elevating/lowering parts 281 and the elevating/lowering part drive part 282 correspond to the mounting table drive part in the present invention. Although FIG. 5 shows an example comprising six elevating/lowering parts 281, the number of elevating/lowering parts 281 is not limited to this, and may be three or more. Furthermore, even in the configuration as shown in FIG. 5, the mounting table 220 may be brought into a horizontal posture as appropriate during stirring. When bringing the mounting table 220 into a horizontal posture, the elevating/lowering part drive part 282 is controlled so that the upper ends of all the elevating/lowering parts 281 are at the same height position.

### Embodiment 2

A second embodiment for carrying out the present invention will be described with reference to FIG. 6. FIG. 6 is a diagram showing a schematic configuration of a stirring apparatus according to the present embodiment. In this figure as well, components that are the same as or correspond to those shown in FIG. 1 are denoted by reference numerals whose last two digits are common, and a description thereof will be omitted as appropriate. The stirring apparatus according to the present embodiment comprises a mounting table 320 on which a Petri dish 310 is mounted, a mounting table support part 330 that supports the mounting table 320 in a state of being inclined with respect to a horizontal direction, and a rotational drive part 350 that rotates the mounting table support part 330. Here, the mounting table support part 330 and the rotational drive part 350 cooperate to function as a mounting table rotating part in the present invention.

The mounting table 320 is a plate-shaped member, and a circular recessed part 323 for fitting the Petri dish 310 is provided in the center of an upper surface thereof (that is, a mounting surface 322).

The mounting table support part 330 is a columnar member extending along a central axis 331 that is inclined with respect to a vertical direction. An end surface orthogonal to the central axis 331 is provided at an upper end of the mounting table support part 330, and the end surface is fixed to the center of a lower surface of the mounting table 320. As a result, the mounting table 320 is supported by the mounting table support part 330 in a state of being inclined by a predetermined inclination angle with respect to the horizontal direction. Here, it is desirable that the inclination angle be, for example, 0° to 30°. On the other hand, a lower end of the mounting table support part 330 is connected to the rotational drive part 350.

The rotational drive part 350 includes a drive mechanism such as a motor (not shown), and autorotates the mounting table support part 330 about its central axis 331.

When the stirring apparatus according to the present embodiment is used, as in Embodiment 1 described above, a Petri dish 310 housing a liquid 311 to be stirred is fitted into the recessed part 323 of the mounting table 320. As described above, since the mounting table 320 is in a state of being inclined with respect to the horizontal plane, inside the Petri dish 310 mounted on the mounting table 320, the liquid 311 is in a state of being pressed against an inner peripheral surface of the Petri dish 310 by gravity. Thereafter, when the user operates a switch or the like (not shown) to actuate the rotational drive part 350, the mounting table support part 330 rotates forwardly or reversely, and as a result, the liquid 311 in the Petri dish 310 is stirred by a shearing effect. Here, it is desirable that a period of rotation of the mounting table support part 330 be, for example, 0.5 seconds to 3 seconds. In the present embodiment as well, the forward rotation or reverse rotation of the mounting table support part 330 may be alternately repeated at predetermined time intervals. Furthermore, in the present embodiment as well, a vertical drive mechanism 170 as shown in FIG. 4 may be provided, and the mounting table 320 may be brought into a horizontal posture as appropriate during stirring.

Although the embodiments for carrying out the present invention have been described above with specific examples, the present invention is not limited to Embodiment 1 or Embodiment 2 described above, and appropriate modifications are permitted within the spirit of the present invention.

For example, although the liquids 111 and 311 in the Petri dishes 110, 210, and 310 are described as the object to be stirred above, the object to be stirred may be anything that has fluidity, and may be, for example, a particulate or powdery solid, or a combination of such a solid and a liquid.

Furthermore, the container for housing the object to be stirred is not limited to a dish-shaped container such as the Petri dishes 110, 210, and 310, and may be, for example, a bottle-shaped container such as a vial, or a bottomed tubular container such as a test tube or a microtube. Furthermore, the container in the present invention may be one provided with a plurality of parts for housing the object to be stirred, such as a multi-well plate.

Furthermore, although in the above description, the Petri dishes 110, 210, and 310 are fitted into the recessed parts 123, 223, and 323 provided in the mounting tables 120, 220, and 320 so that the Petri dishes 110, 210, and 310 do not slide off the inclined mounting tables 120, 220, and 320, the present invention is not limited to this. For example, a container such as the Petri dish 110, 210, or 310 may be held by a holding means such as a clip provided on the mounting surface 122, 222, or 322, or an outer peripheral surface of a container such as the Petri dish 110, 210, or 310 may be pressed from a plurality of directions by a pressing means such as a ball plunger, thereby fixing the container on the mounting table 120, 220, or 320. Furthermore, a degree of mixing of the object to be stirred may be made changeable by changing a rotational speed or rotational acceleration of the mounting table support part 130 or 330 by the rotational drive part 150 or 350, or an elevating/lowering speed of the elevating/lowering part 281 by the elevating/lowering part drive part 282.

### [Aspects]

It will be apparent to those skilled in the art that the exemplary embodiments described above are specific examples of the following aspects.

(Item 1) A stirring apparatus according to one aspect of the present invention comprises:
a mounting table provided with a mounting surface on which a container housing an object to be stirred is mounted; and
a mounting table drive part that supports the mounting table in a state where the mounting surface is inclined with respect to a horizontal direction, and that continuously changes a direction of inclination of the mounting surface.

(Item 2) The stirring apparatus according to item 1, wherein
the mounting table drive part comprises:
a mounting table support part having a mounting table support surface that is inclined with respect to the horizontal direction and on which the mounting table is mounted;
a connecting part that is interposed between the mounting table and the mounting table support surface and that connects both so as to be slidably rotatable;
a rotational drive part that autorotates the mounting table support part about a vertical axis passing through the mounting table support surface; and
a rotation restricting part that restricts rotation of the mounting table while allowing a change in the direction of inclination of the mounting table.

(Item 3) The stirring apparatus according to item 1, wherein
the mounting table drive part comprises:
three or more elevating/lowering parts connected to the mounting table at positions spaced apart from each other in the horizontal direction; and
an elevating/lowering part drive part that individually elevates and lowers each of the three or more elevating/lowering parts.

(Item 4) A stirring apparatus comprises:
a mounting table provided with a mounting surface on which a container housing an object to be stirred is mounted; and
a mounting table rotating part that autorotates the mounting table about an axis orthogonal to the mounting surface in a state where the mounting surface is inclined with respect to a horizontal direction.

(Item 5) The stirring apparatus according to any one of items 1 to 4, wherein the container is a Petri dish.

### REFERENCE SIGNS LIST

110, 210, 310 . . . Petri dish
111, 311 . . . Liquid
120, 220, 320 . . . Mounting table
122, 222, 322 . . . Mounting surface
123, 223, 323 . . . Recessed part
124 . . . Engaging part
130, 330 . . . Mounting table support part
131, 331 . . . Central axis of mounting table support part
132 . . . Support surface
140 . . . Connecting part
150, 350 . . . Rotational drive part
160 . . . Rotation restricting part
281 . . . Elevating/lowering part
282 . . . Elevating/lowering part drive part

1. A stirring apparatus, comprising:
a mounting table provided with a mounting surface on which a container housing an object to be stirred is mounted; and
a mounting table drive part that supports the mounting table in a state where the mounting surface is inclined with respect to a horizontal direction, and that continuously changes a direction of inclination of the mounting surface.

2. The stirring apparatus according to claim 1, wherein the mounting table drive part comprises:
a mounting table support part having a mounting table support surface that is inclined with respect to the horizontal direction and on which the mounting table is mounted;
a connecting part that is interposed between the mounting table and the mounting table support surface and that connects both so as to be slidably rotatable;
a rotational drive part that autorotates the mounting table support part about a vertical axis passing through the mounting table support surface; and
a rotation restricting part that restricts rotation of the mounting table while allowing a change in the direction of inclination of the mounting table.

3. The stirring apparatus according to claim 1, wherein the mounting table drive part comprises:
three or more elevating/lowering parts connected to the mounting table at positions spaced apart from each other in the horizontal direction; and
an elevating/lowering part drive part that individually elevates and lowers each of the three or more elevating/lowering parts.

4. A stirring apparatus, comprising:
a mounting table provided with a mounting surface on which a container housing an object to be stirred is mounted; and
a mounting table rotating part that autorotates the mounting table about an axis orthogonal to the mounting surface in a state where the mounting surface is inclined with respect to a horizontal direction.

5. The stirring apparatus according to claim 1, wherein the container is a Petri dish.
